(19) United States
(12) Patent Application Publication
(10) Pub. No.: US 2016/0347699 A1
(43) Pub. Date: Dec. 1, 2016

(54) METHOD FOR PRODUCING 5-NORBORNENE-2-SPIRO-ALPHA-CYCLOALKANONE- ALPHA'-SPIRO-2''-5''-NORBORNENE

(71) Applicant: JX NIPPON OIL & ENERGY CORPORATION, Tokyo (JP)

(72) Inventors: Rieko FUJISHIRO, Tokyo (JP); Shinichi KOMATSU, Tokyo (JP); Takeshi KOIKE, Tokyo (JP)

(73) Assignee: JX NIPPON OIL & ENERGY CORPORATION, Tokyo (JP)

(21) Appl. No.: 15/112,741

(22) PCT Filed: Jan. 9, 2015
(86) PCT No.: PCT/JP2015/050463

§ 371 Date: Jul. 20, 2016

(30) Foreign Application Priority Data
Jan. 20, 2014 (JP) 2014-008081

(51) Int. Cl. C07C 45/68

(52) U.S. Cl. CPC C07C 45/68; C07C 2103/94

(57) ABSTRACT

A method for producing a 5-norbornene-2-spiro-α-cycloalkanone-α′-spiro-2″-5″-norbornene, comprising: a first step of forming a specific Mannich base by reacting a specific carbonyl compound and a specific amine compound with each other in an acidic solvent comprising a formaldehyde derivative and an acid represented by a formula: HX (in the formula, X represents F or the like), to thereby obtain a reaction liquid comprising the Mannich base in the acidic solvent; and a second step of reacting the Mannich base and a specific diene compound with each other by adding an organic solvent, a base in an amount of 1.0 to 20.0 mole equivalents to the acid, and the diene compound to the reaction liquid, and then heating the reaction liquid, to thereby form a specific 5-norbornene-2-spiro-α-cycloalkanone-α′-spiro-2″-5″-norbornene, wherein a content of the acid in the acidic solvent used in the first step is 0.01 to 0.075 mole equivalents to the ketone group of the carbonyl compound.

### TECHNICAL FIELD

The present invention relates to a method for producing a 5-norbornene-2-spiro-α-cycloalkanone-α′-spiro-2″-5″-norbornene.

### BACKGROUND ART

Conventionally, a wholly aromatic polyimide (trade name “Kapton”) has been known as a material necessary for cutting-edge industries for aerospace and aviation applications and the like. However, the wholly aromatic polyimide becomes brown in color, because intramolecular charge transfer (CT) occurs between a tetracarboxylic dianhydride unit of an aromatic ring system and a diamine unit of another aromatic ring system. Hence, the wholly aromatic polyimide cannot be used in optical applications and the like, where transparency is necessary. For this reason, alicyclic polyimides which do not undergo the intramolecular CT and which have high light transmittance have attracted attention recently, and various compounds (raw material compounds and the like) usable for producing the alicyclic polyimides have been developed.

In general, alicyclic tetracarboxylic dianhydrides have been used for producing the alicyclicpolyimides. In addition, as a compound which can be preferably used for producing the alicyclic tetracarboxylic dianhydrides and a method for producing the compound, for example, International Publication No. WO2011/099517 (PTL 1) discloses a 5-norbornene-2-spiro-α-cycloalkanone-α′-spiro-2″-5″-norbornene represented by a specific general formula and a method for producing the compound. In addition, PTL 1 also discloses that an alicyclic polyimide having a high light transmittance and a sufficiently high heat resistance can be produced when an alicyclic tetracarboxylic dianhydride is formed by using the 5-norbornene-2-spiro-α-cycloalkanone-α′-spiro-2″-5″-norbornene, and then an alicyclic polyimide is produced. Note that, according to the method for producing a 5-norbornene-2-spiro-α-cycloalkanone-α′-spiro-2″-5″-norbornene as described in PTL 1, the norbornene can be produced sufficiently efficiently in a sufficiently high yield, and the method for producing a norbornene described in PTL 1 above is an industrially applicable method. However, the advent of a production method which makes it possible to efficiently obtain such a compound in a higher yield has been awaited from the viewpoint of industrially producing a larger amount of a 5-norbornene-2-spiro-α-cycloalkanone-α′-spiro-2″-5″-norbornene.

### CITATION LIST

### Patent Literature

[PTL 1] International Publication No. WO2011/099517

### SUMMARY OF INVENTION

### Technical Problem

The present invention has been made in view of the problems of the conventional technologies, and an object of the present invention is to provide a method for producing a 5-norbornene-2-spiro-α-cycloalkanone-α′-spiro-2″-5″-norbornene, the method making it possible to more efficiently produce the 5-norbornene-2-spiro-α-cycloalkanone-α′-spiro-2″-5″-norbornene in a higher yield.

### Solution to Problem

The present inventors have conducted earnest study to achieve the above-described object, and consequently found that a 5-norbornene-2-spiro-α-cycloalkanone-α′-spiro-2″-5″-norbornene can be produced more efficiently in a higher yield by a method for producing a 5-norbornene-2-spiro-α-cycloalkanone-α′-spiro-2″-5″-norbornene, the method comprising: a first step of forming a Mannich base represented by the general formula (3) shown below by reacting a carbonyl compound represented by the general formula (1) shown below and an amine compound represented by the general formula (2) shown below with each other in an acidic solvent comprising a formaldehyde derivative and an acid represented by a formula: HX (in the formula, X represents one selected from the group consisting of F, Cl, Br, I, CH₃COO, CF₃COO, CH₃SO₃, CF₃SO₃, C₆H₅SO₃, CH₃C₆H₄SO₃, HOSO₃, and H₂PO₄), to thereby obtain a reaction liquid comprising the Mannich base in the acidic solvent; and

a second step of reacting the Mannich base and a diene compound represented by the general formula (4) shown below with each other by adding an organic solvent, a base in an amount of 1.0 to 20.0 mole equivalents to the acid, and the diene compound to the reaction liquid, and then heating the reaction liquid, to thereby form a 5-norbornene-2-spiro-α-cycloalkanone-α′-spiro-2″-5″-norbornene represented by the general formula (5) shown below, wherein

a content of the acid in the acidic solvent used in the first step is 0.01 to 0.075 mole equivalents to the ketone group of the carbonyl compound. This finding has led to the completion of the present invention.

Specifically, a method for producing a 5-norbornene-2-spiro-α-cycloalkanone-α′-spiro-2″-5″-norbornene of the present invention comprises:

a first step of forming a Mannich base by reacting a carbonyl compound and an amine compound with each other in an acidic solvent, to thereby obtain a reaction liquid comprising the Mannich base in the acidic solvent,
the acidic solvent comprising a formaldehyde derivative and an acid represented by a formula: HX (in the formula, X represents one selected from the group consisting of F, Cl, Br, I, CH₃COO, CF₃COO, CH₃SO₃, CF₃SO₃, C₆H₅SO₃, CH₃C₆H₄SO₃, HOSO₃, and H₂PO₄),
the carbonyl compound being represented by the following general formula (1):

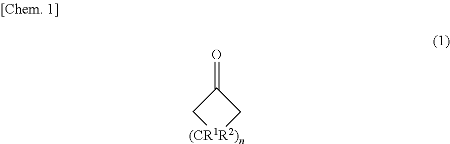

[in the formula (1), R¹ and R² each independently represent one selected from the group consisting of a hydrogen atom, alkyl groups having 1 to 10 carbon atoms, and a fluorine atom, and n represents an integer of 0 to 12],
the amine compound being represented by the following general formula (2):

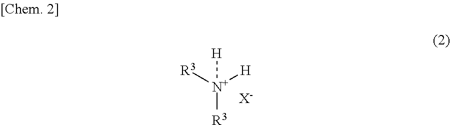

[in the formula (2), R³s each independently represent one selected from the group consisting of linear chain saturated hydrocarbon groups having 1 to 20 carbon atoms, branched chain saturated hydrocarbon groups having 3 to 20 carbon atoms, saturated cyclic hydrocarbon groups having 3 to 20 carbon atoms, and saturated hydrocarbon groups having a hydroxyl group and 1 to 10 carbon atoms, the two R³s may be bonded to each other to form a ring selected from the group consisting of a pyrrolidine ring, a piperidine ring, a piperazine ring, and a morpholine ring, and X⁻ represents one selected from the group consisting of F⁻, Cl⁻, Br⁻, I⁻, CH₃COO⁻, CF₃COO⁻, CH₃SO₃⁻, CF₃SO₃⁻, C₆H₅SO₃⁻, CH₃C₆H₄SO₃⁻, HOSO₃⁻, and H₂PO₄⁻],
the Mannich base being represented by the following general formula (3):

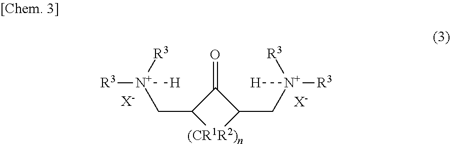

[R¹, R², and n in the formula (3) have the same meanings as those of R¹, R², and n in the formula (1), and R³s and X⁻s in the formula (3) have the same meanings as those of R³s and X⁻ in the formula (2)]; and

a second step of reacting the Mannich base and a diene compound with each other by adding an organic solvent, a base in an amount of 1.0 to 20.0 mole equivalents to the acid, and the diene compound to the reaction liquid, and then heating the reaction liquid, to thereby form a 5-norbornene-2-spiro-α-cycloalkanone-α′-spiro-2″-5″-norbornene,
the diene compound being represented by the following general formula (4):

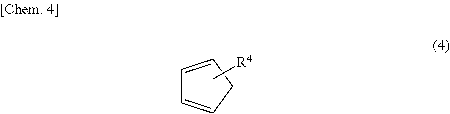

[in the formula (4), R⁴ represents one selected from the group consisting of a hydrogen atom, alkyl groups having 1 to 10 carbon atoms, and a fluorine atom],
the 5-norbornene-2-spiro-α-cycloalkanone-α′-spiro-2″-5″-norbornene being represented by the following general formula (5):

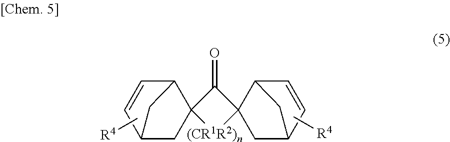

[R¹, R², and n in the formula (5) have the same meanings as those of R¹, R², and n in the formula (1), and R⁴s in the formula (5) have the same meaning as that of R⁴ in the formula (4)], wherein

a content of the acid in the acidic solvent used in the first step is 0.01 to 0.075 mole equivalents to the ketone group of the carbonyl compound.

In the method for producing a 5-norbornene-2-spiro-α-cycloalkanone-α′-spiro-2″-5″-norbornene of the present invention, the content of the acid in the acidic solvent used in the first step is more preferably 0.01 to 0.070 mole equivalents to the ketone group of the carbonyl compound.

### Advantageous Effects of Invention

According to the present invention, it is possible to provide a method for producing a 5-norbornene-2-spiro-α-cycloalkanone-α′-spiro-2″-5″-norbornene, the method making it possible to more efficiently produce the 5-norbornene-2-spiro-α-cycloalkanone-α′-spiro-2″-5″-norbornene in a higher yield.

### BRIEF DESCRIPTION OF DRAWING

FIG. 1 is a graph showing the relationship between the reaction yield of a product obtained in each of Examples 1 to 4 and Comparative Example 2 and 3 and the content (mole equivalents) of an acid (HCl) in a first mixture liquid used for producing the product.

### DESCRIPTION OF EMBODIMENTS

Hereinafter, the present invention will be described in detail based on preferred embodiments thereof.

A method for producing a 5-norbornene-2-spiro-α-cycloalkanone-α′-spiro-2″-5″-norbornene of the present invention comprises:

a first step of forming a Mannich base represented by the general formula (3) by reacting a carbonyl compound represented by the general formula (1) and an amine compound represented by the general formula (2) with each other in an acidic solvent comprising a formaldehyde derivative and an acid represented by the formula: HX, to thereby obtain a reaction liquid comprising the Mannich base in the acidic solvent; and

a second step of reacting the Mannich base and a diene compound represented by the general formula (4) with each other by adding an organic solvent, a base in an amount of 1.0 to 20.0 mole equivalents to the acid, and the diene compound to the reaction liquid, and then heating the reaction liquid, to thereby form a 5-norbornene-2-spiro-α-cycloalkanone-α′-spiro-2″-5″-norbornene represented by the general formula (5), wherein a content of the acid in the acidic solvent used in the first step is 0.01 to 0.075 mole equivalents to the ketone group of the carbonyl compound. Hereinafter, the steps are described separately. Note that, in the following description, the 5-norbornene-2-spiro-α-cycloalkanone-α′-spiro-2″-5″-norbornene represented by the general formula (5) is simply referred to as “bis(spiro norbornene)” in some cases.

(First Step)

The first step is a step of forming a Mannich base represented by the general formula (3) by reacting a carbonyl compound represented by the general formula (1) and an amine compound represented by the general formula (2) with each other in the acidic solvent, to thereby obtain a reaction liquid comprising the Mannich base in the acidic solvent. Note that, in this first step, a content of the acid in the acidic solvent is 0.01 to 0.075 mole equivalents to the ketone group of the carbonyl compound.

The acidic solvent used in this first step comprises a formaldehyde derivative. The formaldehyde derivative is not particularly limited, as long as the formaldehyde derivative can be used for producing a so-called Mannich base. It is possible to use, as appropriate, any of known compounds capable of supplying “formaldehyde” used for producing a Mannich base into a reaction system (for example, formaldehyde itself, as well as compounds capable of supplying formaldehyde into the acidic solvent by decomposition in the acidic solvent, and the like). As the compound capable of supplying formaldehyde into the reaction system, for example, formaldehyde, a cyclic derivative of formaldehyde (trioxane, 1,3-dioxolan, or the like), a polymeric derivative of formaldehyde (for example, paraformaldehyde or the like) can be used, as appropriate. Examples of the formaldehyde derivatives include formalin, paraformaldehyde, trioxane, 1,3-dioxolan, 1,3-dioxole, 1,3-dioxane, 1,3-dioxin, 1,3-dioxepane, dihydro-1,3-dioxepin, 1,3-dioxepin, 1,3-dioxocane, dihydro-1,3-dioxocin, 1,3-dioxocin, formaldehyde dimethyl acetal, formaldehyde diethyl acetal, formaldehyde dipropyl acetal, formaldehyde dibutyl acetal, formaldehyde diphenyl acetal, and the like.

In addition, of these formaldehyde derivatives, formalin, paraformaldehyde, trioxane, and 1,3-dioxolan are preferable, and formalin and paraformaldehyde are more preferable, from the viewpoint of availability. In addition, one of these formaldehyde derivatives alone or a combination of two or more thereof may be used. However, it is preferable to use one of these formaldehyde derivatives alone from the viewpoint of purification.

The content of the formaldehyde derivative in the acidic solvent is preferably 2.0 to 50.0% by mass, and more preferably 4.0 to 25.0% by mass. If the content of the formaldehyde derivative is less than the lower limit, the yield of the Mannich base represented by the general formula (3) tends to be low. Meanwhile, if the content exceeds the upper limit, the yield tends to be low, and purification tends to be difficult.

In addition to the formaldehyde derivative, the acidic solvent used in the first step also comprises an acid represented by a formula: HX (in the formula, X represents one selected from the group consisting of F, Cl, Br, I, CH₃COO, CF₃COO, CH₃SO₃, CF₃SO₃, C₆H₅SO₃, CH₃C₆H₄SO₃, HOSO₃, and H₂PO₄).

The kind of the acid (HX) is not particularly limited, as long as the acid is represented by the above-described formula: HX. From the viewpoint of the stability of the Mannich base represented by the general formula (3) in the acidic solvent, an acid whose X in the formula is F, Cl, Br, CH₃COO, or CF₃COO is more preferable, and an acid whose X in the formula is Cl or CH₃COO is further preferable.

In the acidic solvent, the content of the acid (HX) needs to be 0.01 to 0.075 mole equivalents to the ketone group of the carbonyl compound represented by the general formula (1). If the content of the acid is less than the lower limit, it is difficult to produce iminium ions efficiently, so that the Mannich base cannot be produced efficiently at a sufficiently high level. As a result, the bis(spiro norbornene) cannot be produced in a sufficiently high yield. Meanwhile, if the content of the acid exceeds the upper limit, it is difficult to produce the bis(spiro norbornene) in a sufficiently high yield. Note that the mole equivalence of the acid can be obtained by calculating a value of the total amount by mole of the acid in the reaction system relative to the total amount by mole of the ketone group of the carbonyl compound in the reaction system ([the total amount by mole of the acid]/[the total amount by mole of the ketone group (═C═O)]).

In addition, the content of the acid (HX) in the acidic solvent is more preferably 0.01 to 0.070 mole equivalents, and further preferably 0.012 to 0.050 mole equivalents to the ketone groups of the carbonyl compound represented by the general formula (1). With such a content of the acid (HX), the bis(spiro norbornene) tends to be produced in a higher yield.

In addition, the content ratio of the acid (HX) in the acidic solvent is preferably 0.01 mol/L or higher (more preferably 0.01 to 0.4 mol/L, and further preferably 0.02 to 0.2 mol/L). If the content ratio of the acid is lower than the lower limit, the yield of the Mannich base prepared in the first step tends to be so insufficient that the bis(spiro norbornene) represented by the general formula (1) cannot be prepared sufficiently and efficiently. Meanwhile, if the content ratio of the acid (HX) exceeds the upper limit, the yield tends to be low, and the purification tends to be difficult.

Moreover, the acidic solvent may comprise a solvent in addition to the formaldehyde derivative and the acid. Examples of the solvent include water, alcohols, glycols, ethylene glycol, glycerin, ethers, cellosolves, nitriles, amides, methylcyclohexane, and the like.

In addition, regarding the solvent which may be contained in the acidic solvent, it is preferable that an organic solvent having a boiling point temperature of 85 to 110° C. and being incapable of dissolving the Mannich base be contained (hereinafter, this organic solvent is simply referred to as “first organic solvent” in some cases), from the viewpoint of suppressing formation of by-products in producing the Mannich base. The use of the first organic solvent makes it possible to easily control the reaction temperature to a temperature near the temperature range of the boiling point of the first organic solvent, when the reaction is allowed to proceed in the acidic solvent. This makes it possible to suppress rapid increase in the temperature of the acidic solvent due to rapid generation of heat of reaction, and to more sufficiently suppress the formation of by-products, which are more likely to be produced at a higher temperature. Consequently, the Mannich base tends to be produced more efficiently. Note that, in a case where a reactor with a larger capacity is used, it tends to be more difficult to control the reaction temperature against rapid generation of heat of reaction than in a case where a reactor with a smaller capacity is used. However, the use of the first organic solvent makes it possible to easily control the heating temperature to a temperature near the temperature range of the boiling point of the first organic solvent. Hence, when a reactor with a larger capacity is used, the use of the first organic solvent is particularly preferable. Note that, if the boiling point of the first organic solvent is lower than the lower limit, the liquid temperature during the reaction tends not to reach an optimum temperature, so that the formation of the Mannich base tends to be insufficient, and the yields tend be low. Meanwhile, if the boiling point of the first organic solvent exceeds the upper limit, the obtained effect of suppressing the increase in temperature tends to be insufficient, and it is difficult to suppress the formation of by-products always sufficiently, and the yield tends to be low. In addition, the expression “being incapable of dissolving the Mannich base” herein means that the solubility of the Mannich base represented by the general formula (3) in the organic solvent under a condition of 20 to 65° C. is lower than 1% by weight. Moreover, the boiling point temperature of the first organic solvent is a boiling point temperature under a condition where the pressure is normal pressure (0.1 MPa).

In addition, the organic solvent having a boiling point temperature of 85 to 110° C. and being incapable of dissolving the Mannich base is preferably a hydrocarbon-based solvent having 3 to 20 carbon atoms (more preferably 3 to 10 carbon atoms). If the number of carbon atoms is less than the lower limit, the organic solvent tends to be gas at normal temperature and normal pressure. Meanwhile, if the number of carbon atoms exceeds the upper limit, the organic solvent tends to be solid at normal temperature and normal pressure. The hydrocarbon-based solvent having 3 to 20 carbon atoms is more preferably a saturated hydrocarbon optionally having a side chain of a hydrocarbon group. Especially, methylcyclohexane, isoparaffin-based hydrocarbons having 6 to 20 carbon atoms, cyclohexane, and n-heptane are further preferable. Examples of the isoparaffin-based hydrocarbons include 2-methylheptane, 2,2,4-trimethylpentane, and the like. In addition, commercially available products may be used as the isoparaffin-based hydrocarbons. For example, one manufactured by Idemitsu Kosan Company, Limited under the trade name of “IP Solvent” or the like may be used, as appropriate. Moreover, as the isoparaffin-based hydrocarbon, 2,2,4-trimethylpentane or IP Solvent (trade name, manufactured by Idemitsu Kosan Company, Limited) is preferably used from the viewpoint of availability. In other words, the hydrocarbon-based solvent having 3 to 20 carbon atoms is particularly preferably methylcyclohexane, IP Solvent (trade name, manufactured by Idemitsu Kosan Company, Limited), cyclohexane, n-heptane, or 2,2,4-trimethylpentane. Note that one of these solvents alone or a combination of two or more thereof may be used.

In addition, the content of the solvent (when two or more solvents are contained, the total amount of the solvents) in the acidic solvent is preferably 20 to 60% by mass, and more preferably 30 to 50% by mass. If the content of the solvent is less than the lower limit, the mixing tends to be non-uniform, and the yield of the Mannich base tends to be low. Meanwhile, if the content of the solvent exceeds the upper limit, the reaction rate tends to be low, and the yield tends to decrease.

In addition, when the first organic solvent is used, the content of the first organic solvent in the acidic solvent is preferably 5 to 30% by mass, and more preferably 10 to 20% by mass. If the content of the first organic solvent is less than the lower limit, the effect of suppressing the increase in temperature tends not to be obtained sufficiently, it tends to be difficult to suppress the formation of by-product always sufficiently, and the yield tends to be low. Meanwhile, if the content of the first organic solvent exceeds the upper limit, the first organic solvent tends to cause decrease in yield of the target compound in a purification step.

The acidic solvent comprising the formaldehyde derivative and the acid (an acid represented by the formula: HX) in the range from 0.01 to 0.075 mole equivalents to the ketone group of the carbonyl compound represented by the general formula (1) is used in the first step. The use of this acidic solvent further makes it possible to react the carbonyl compound and the amino compound with each other under an acidic condition, where the acid is present in excess. Consequently, it is possible to efficiently produce the Mannich base represented by the general formula (3), which is a reaction intermediate used for preparation of bis(spiro norbornene)s.

In addition, the carbonyl compound used in the first step is represented by the following general formula (1)

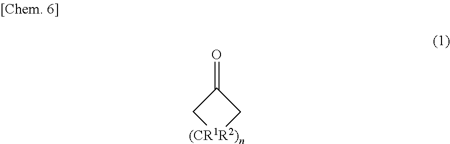

[in the formula (1), R¹ and R² each independently represent one selected from the group consisting of a hydrogen atom, alkyl groups having 1 to 10 carbon atoms, and a fluorine atom, and n represents an integer of 0 to 12].

In addition, the alkyl group which can be selected as each of R¹ and R² in the general formula (1) is an alkyl group having 1 to 10 carbon atoms. When the number of carbon atoms of the alkyl group exceeds 10, the heat resistance of a polyimide obtained in the use as a monomer of a polyimide decreases. In addition, the number of carbon atoms of the alkyl group which can be selected as each of R¹ and R² is preferably 1 to 5 and more preferably 1 to 3, from the viewpoint that a higher heat resistance can be obtained when a polyimide is produced. In addition, the alkyl group which can be selected as each of R¹ and R² may be linear or branched.

Of the above-described substituents, the substituent which can be selected as each of R¹ and R² in the general formula (1) is preferably a hydrogen atom or an alkyl group having 1 to 10 carbon atoms (more preferably 1 to 5, and further preferably 1 to 3 carbon atoms), and is particularly preferably a hydrogen atom or a methyl group, from the viewpoint of ease of the purification. In addition, when multiple R¹s are present in the general formula (1) (when n is 2 or greater), the multiple R's may be the same or different, and are preferably the same from the viewpoints of ease of purification and the like. In addition, when multiple R² are present in the general formula (1) (when n is 2 or greater), the multiple R²s may be the same or different, and are preferably the same from the viewpoints of ease of purification and the like. Moreover, R¹ and R² in the general formula (1) are more preferably the same from the viewpoints of ease of purification and the like.

In the general formula (1), n represents an integer of 0 to 12. If the value of n is greater than the upper limit, it is difficult to purify the bis(spiro norbornene) represented by the general formula (1). In addition, an upper limit value of the numeric value range of n in the general formula (1) is more preferably 5, and particularly preferably 3, from the viewpoint that the bis(spiro norbornene) becomes easier to purify and from other viewpoints. Meanwhile, a lower limit value of the numeric value range of n in the general formula (1) is more preferably 1, and particularly preferably 2, from the viewpoint of the stability of the raw material. Accordingly, n in the general formula (1) is particularly preferably an integer of 2 or 3. As the carbonyl compound represented by the general formula (1), for example, any of the carbonyl compounds (cyclopropanone, cyclobutanone, cyclopentanone, cyclohexanone, cycloheptanone, cyclooctanone, and the like) listed as examples in International Publication No. WO2011/099517 may be used, as appropriate.

In addition, a method for preparing such a carbonyl compound represented by the general formula (1) is not particularly limited, and a known method can be employed, as appropriate. In addition, it is also possible to use commercially available one as the compound represented by the general formula (1).

Meanwhile, the amine compound used in the first step is represented by the following general formula (2):

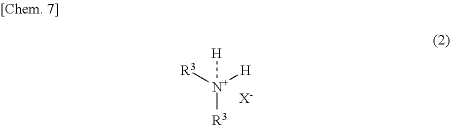

[in the formula (2), R³s each independently represent any one selected from the group consisting of linear chain saturated hydrocarbon groups having 1 to 20 carbon atoms, branched chain saturated hydrocarbon groups having 3 to 20 carbon atoms, saturated cyclic hydrocarbon groups having 3 to 20 carbon atoms, and saturated hydrocarbon groups having a hydroxyl group and 1 to 10 carbon atoms, the two R³s may be bonded to each other to form a ring selected from the group consisting of a pyrrolidine ring, a piperidine ring, a piperazine ring, and a morpholine ring, and X⁻ represents one selected from the group consisting of F⁻, Cl⁻, Br⁻, I⁻, CH₃COO⁻, CF₃COO⁻, CH₃SO₃⁻, CF₃SO₃⁻, C₆H₅SO₃⁻, CH₃C₆H₄SO₃⁻, HOSO₃⁻, and H₂PO₄⁻].

The linear chain saturated hydrocarbon group which can be selected as each R³ in the general formula (2) is one having 1 to 20 carbon atoms. The linear chain saturated hydrocarbon group has more preferably 1 to 10 carbon atoms, and further preferably 1 to 5 carbon atoms. If the number of carbon atoms of the linear chain saturated hydrocarbon group exceeds the upper limit, the purification tends to be difficult. The linear chain saturated hydrocarbon group which can be selected as each R³ is more preferably a methyl group or an ethyl group, from the viewpoint of ease of the purification.

Meanwhile, the branched chain saturated hydrocarbon group which can be selected as each R³ is one having 3 to 20 carbon atoms. The branched chain saturated hydrocarbon group has more preferably 3 to 10 carbon atoms, and further preferably 3 to 5 carbon atoms. If the number of the carbon atoms of the branched chain saturated hydrocarbon group exceeds the upper limit, the purification tends to be difficult. The branched chain saturated hydrocarbon group which can be selected as each R³ is more preferably an isopropyl group, from the viewpoint of ease of the purification.

Meanwhile, the saturated cyclic hydrocarbon group which can be selected as each R³ is one having 3 to 20 carbon atoms. The saturated cyclic hydrocarbon group has more preferably 3 to 10 carbon atoms, and further preferably 5 to 6 carbon atoms. If the number of the carbon atoms of the saturated cyclic hydrocarbon group exceeds the upper limit, the purification becomes difficult. Meanwhile, if the number of the carbon atoms is less than the lower limit, the chemical stability tends to be lowered. The saturated cyclic hydrocarbon group which can be selected as each R³ is more preferably a cyclopentyl group or a cyclohexyl group from the viewpoints of ease of purification and of chemical stability.

The saturated hydrocarbon group having a hydroxyl group which can be selected as each R³ is one in which the hydrocarbon group has 1 to 10 carbon atoms. In the saturated hydrocarbon group having a hydroxyl group, the number of carbon atoms is more preferably 2 to 10, and further preferably 2 to 5. If the number of carbon atoms of the saturated hydrocarbon group having a hydroxyl group exceeds the upper limit, the purification becomes difficult. Meanwhile, if the number of the carbon atoms is less than the lower limit, the chemical stability tends to be poor. The saturated hydrocarbon group having a hydroxyl group which can be selected as each R³ is more preferably a 2-hydroxyethyl group from the viewpoints of ease of purification and of chemical stability.

In addition, the two R³s in the general formula (2) may be bonded to each other to form any ring of a pyrrolidine ring, a piperidine ring, a piperazine ring, and a morpholine ring. Specifically, regarding the two R³s in the general formula (2), R³s may be bonded to each other to form a pyrrolidine ring, a piperidine ring, a piperazine ring, or a morpholine ring together with the nitrogen atom (N) in the formula (2). When the R³s are bonded to each other to form a ring as described above, the ring is more preferably a morpholine ring from the viewpoint of the odor.

Moreover, as R³s in the general formula (2), a methyl group, an ethyl group, a 2-hydroxyethyl group, or morpholine is more preferable from the viewpoint of ease of the purification. In addition, when the two R³ in the general formula (2) does not form a ring, the two R³ are preferably the same from the viewpoint of availability.

X⁻ in the general formula (2) is a so-called counter anion. X⁻ in the formula (2) is any one selected from the group consisting of F⁻, Cl⁻, Br⁻, I⁻, CH₃COO⁻, CF₃COO⁻, CH₃SO₃⁻, CF₃SO₃⁻, C₆H₅SO₃⁻, CH₃C₆H₄SO₃⁻, HOSO₃⁻, and H₂PO₄⁻. X⁻ is preferably F⁻, C₁⁻, Br⁻, CH₃COO⁻, or CF₃COO⁻, and more preferably Cl⁻ or CH₃COO⁻ from the viewpoint of the stability of the obtained Mannich base represented by the general formula (3).

In addition, as the amine compound represented by the general formula (2), for example, any of the amine compounds listed as examples in International Publication No. WO2011/099517 (salts of secondary amines such as dimethylamine, diethylamine, and di-n-propylamine, and the like) may be used, as appropriate. In addition, a method for producing the amine compound is not particularly limited, and a known method can be used, as appropriate. Alternatively, a commercially available product may be used as the amine compound.

In addition, in the first step, the carbonyl compound represented by the general formula (1) and the amine compound represented by the general formula (2) are reacted with each other in the acidic solvent. The amount of the carbonyl compound used for the reaction is such that the concentration thereof in the acidic solvent is preferably 0.01 to 5.0 mol/L, and more preferably 0.1 to 2.0 mol/L. If the amount of the carbonyl compound is less than the lower limit, the production efficiency of the Mannich base represented by the general formula (3) tends to be lowered. Meanwhile, if the amount of the carbonyl compound exceeds the upper limit, a side-reaction product due to a side reaction tends to increase.

In addition, the amount of the amine compound used is preferably 2 mole equivalents or more, and more preferably 2 to 10 mole equivalents to the carbonyl compound. If the amount of the amine compound used is less than the lower limit, the yield of the Mannich base tends to be low. Meanwhile, if the amount of the amine compound used exceeds the upper limit, a side-reaction product due to a side reaction tends to increase.

Note that when a commercially available product is used as the amine compound, and the commercially available product contains the acid (for example, hydrochloric acid or the like) in addition to the amine compound, the amine compound and the acid may be simultaneously supplied into the acidic solvent by supplying the commercially available product into the system. In such a case, the relationship between the amounts of the carbonyl compound and the acid used for the reaction may be adjusted, as appropriate, by measuring, as appropriate, the concentration of the acid in the acidic solvent by a known method, and, if necessary, further adding the acid, or by other method.

In addition, the reaction conditions for reacting the carbonyl compound and the amine compound with each other in the acidic solvent are not particularly limited, and can be changed, as appropriate, according to the kind of the solvent used and the like. An atmosphere with which the acidic solvent is in contact during the reaction is not particularly limited, and is preferably an inert gas atmosphere of nitrogen gas or the like. In addition, the reaction is preferably allowed to proceed under heated conditions from the viewpoint of promoting the reaction. As the heated conditions, it is preferable to employ such heated conditions that the acidic solvent is held at a temperature of 30 to 180° C. (more preferably 80 to 120° C., and further preferably 85 to 110° C.) for 0.5 to 10 hours (more preferably 4 to 8 hours). If the heating temperature (the temperature of the acidic solvent) and/or the heating time are below the lower limits, the yield of the Mannich base represented by the general formula (3) tends to be low. Meanwhile, if the heating temperature and/or the heating time exceed the upper limit, by-products such as bis(vinyl ketone) and vinyl ketone dimer tend to increase, so that the yield of the Mannich base represented by the general formula (3) tends to be low. Note that, from the viewpoints of suppressing the formation of by-products and the like, the heating temperature condition is preferably controlled to a lower temperature within the temperature range. From such a viewpoint, it is preferable that an organic solvent (preferably a hydrocarbon-based solvent having 3 to 20 carbon atoms (more preferably having 3 to 10 carbon atoms)) having a boiling point temperature of 85 to 110° C. and being incapable of dissolving the Mannich base be used as a solvent in the acidic solvent, and the temperature of the reaction system be controlled to a temperature near the boiling point.

Note that a pressure condition during the heating is not particularly limited, and is preferably 0.10 to 10 MPa, and more preferably 0.10 to 1 MPa. If the pressure condition is below the lower limit, the effect of reducing the thermal energy at the solvent recycling tends to be low. Meanwhile, if the pressure condition exceeds the upper limit, such a pressure condition tends to be difficult to achieve because of limitations of equipment.

By reacting the carbonyl compound represented by the general formula (1) and the amine compound represented by the general formula (2) with each other in the presence of the acidic solvent as described above, it is possible to form the Mannich base represented by the following general formula (3):

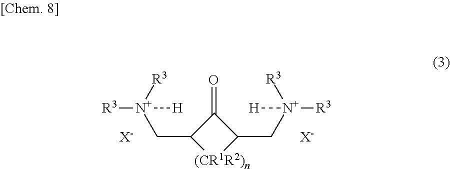

[R¹, R², and n in the formula (3) have the same meanings as those of R¹, R², and n in the formula (1) (preferred ones thereof are also the same), and R³s and X⁻s in the formula (3) have the same meanings as those of R³s and X⁻ in the formula (2) (preferred ones thereof are also the same)]. Thus, a reaction liquid comprising the Mannich base in the acidic solvent can be obtained. Note that multiple R³s in the formula (3) may be the same or different, and are preferably the same from the viewpoint of availability. In addition, multiple X⁻s in the formula (3) may be the same or different, and are preferably the same from the viewpoint of availability.

In addition, the reaction of the carbonyl compound and the amine compound with each other is conducted in the acidic solvent comprising the acid at a ratio of 0.01 to 0.075 mole equivalents to the ketone group of the carbonyl compound used in the reaction in this first step. This makes it possible to form the Mannich base represented by the general formula (3) in a sufficiently high yield. The use of the acidic solvent comprising the acid at such a concentration more sufficiently improves the production efficiency and the yield of the reaction intermediate (Mannich base) in the first step in the present invention. Then, the reaction liquid comprising the thus formed Mannich base is directly used in the second step in the present invention. Hence, the Mannich base can be used efficiently. It is presumed that this aspect also contributes to the improvement in the yield of the final target product.

(Second Step)

The second step is a step of reacting the Mannich base and a diene compound represented by the general formula (4) with each other by adding an organic solvent, a base in an amount of 1.0 to 20.0 mole equivalents to the acid, and the diene compound to the reaction liquid, and then heating the reaction liquid, to thereby form a bis(spiro norbornene) represented by the general formula (5).

In the second step, the reaction liquid obtained in the first step is used. In this manner, the Mannich base is not isolated from the reaction liquid in the second step in the present invention. Hence, the Mannich base, which is the reaction intermediate present in the reaction liquid, can be used highly efficiently, and the step can be simplified. Thus, the bis(spiro norbornene) can be produced sufficiently efficiently.

Moreover, in the second step, the organic solvent is added to the reaction liquid (for convenience, this organic solvent is simply referred to as “second organic solvent” in some cases). The organic solvent is not particularly limited, and organic solvents which can be used for the so-called Diels-Alder reaction can be used as appropriate. Examples of the organic solvents include alcohol-based solvents (including glycol-based solvents, glycerin-based solvents, and other polyol-based solvents), cellosolve-based solvents, ether-based solvents, amide-based solvents, and nitrile-based solvents. A preferred organic solvent can be selected and used, as appropriate, according to the kind of the target bis(spiro norbornene), and the like.

In addition, when the bis(spiro norbornene) is separated and taken out of the reaction liquid by an extraction step after the reaction, the organic solvent used in the second step is preferably an organic solvent immiscible with a saturated hydrocarbon having 5 to 30 carbon atoms, from the viewpoint of simplifying the extraction step. The organic solvent immiscible with a saturated hydrocarbon having 5 to 30 carbon atoms is preferably methanol, methyl cellosolve, dimethylacetamide, dimethyl sulfoxide, ethylene glycol, propylene glycol, 1,3-propanediol, glycerin, propylene glycol monomethyl ether, ethyl cellosolve, dimethylformamide, acetonitrile, and the like. Of these organic solvents, methanol or methyl cellosolve is more preferable from the viewpoint of ease and convenience of the extraction operation. Note that the term “immiscible” herein means that when the organic solvent is added to the reaction liquid at any ratio, the mixture takes a state of two separated layers.

Meanwhile, when the above-described first organic solvent is used for the acidic solvent, and the bis(spiro norbornene) is separated and taken out by a crystallization step after the reaction, the second organic solvent is more preferably an organic solvent in which the solubility of the bis(spiro norbornene) greatly varies depending on the temperature, from the viewpoint of simplifying the crystallization step. As the organic solvent in which the solubility of the bis(spiro norbornene) greatly varies depending on the temperature, for example, an organic solvent can be used preferably in which the solubility of the bis(spiro norbornene) is 5% by weight or more under a condition of 40 to 80° C., while the solubility of the bis(spiro norbornene) is lower than 2% by weight under a condition of −25 to 0° C. The organic solvent is preferably methanol, ethanol, isopropanol, an aqueous solution thereof, or the like. Of these organic solvents, methanol and ethanol are more preferable, from the viewpoint of ease and convenience of the operation for separating the bis(spiro norbornene) and from the viewpoint of the volatility at the drying of the product.

In addition, the amount of the organic solvent (second organic solvent) added to the reaction liquid is not particularly limited, and is preferably 10 to 80% by mass (more preferably 20 to 60% by mass) relative to the total amount of the reaction liquid and the organic solvent (second organic solvent) added. If the concentration of the organic solvent (second organic solvent) is lower than the lower limit, by-products such as vinyl ketone dimer tend to increase, so that the yield of the target product tends to be low. Meanwhile, if the concentration exceeds the upper limit, the reaction rate tends to be low, and hence the yield tends to be low.

In addition, in the second step, a base is added to the reaction liquid. The kind of the base is not particularly limited, and amines, alkali metal hydroxides, and alkaline earth metal hydroxides can be used preferably, from the viewpoint of basicity. Of these bases, dimethylamine, diethylamine, dipropylamine, and dibutylamine are preferable, and dimethylamine is particularly preferable, from the viewpoint of purification.

In addition, the amount of the base added needs to be 1.0 to 20.0 mole equivalents (more preferably 1.0 to 10.0 mole equivalents, and further preferably 1.0 to 5.0 mole equivalents) to the acid contained in the reaction liquid. If the amount of the base added is less than the lower limit, the decomposition of the Mannich base is suppressed, so that the bis(vinyl ketone) intermediate, which serves as a raw material of the target product, becomes difficult to form. Meanwhile, if the amount of the base added exceeds the upper limit, the recovery is difficult because a large amount of a neutralizing agent is necessary during purification or crystallization. As described above, in the present invention, while the reaction liquid is made neutral or basic, the Mannich base and the diene compound are reacted with each other in the second step. Thus, the formation of by-products (for example, a dimerization product (dimer) formed due to dimerization by the hetero Diels-Alder reaction of a bis(vinyl ketone) formed by elimination of an amino compound from the Mannich base) is sufficiently suppressed, and the target bis(spiro norbornene) can be produced in a sufficiently high selectivity.

Moreover, in the second step, a diene compound represented by the following general formula (4) is added to the reaction liquid:

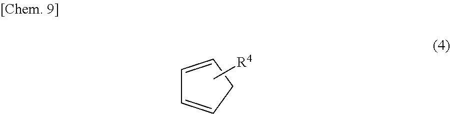

[in the formula (4), R⁴ represents at least one selected from the group consisting of a hydrogen atom, alkyl groups having 1 to 10 carbon atoms, and a fluorine atom].

The alkyl group which can be selected as R⁴ in the general formula (4) is an alkyl group having 1 to 10 carbon atoms. If the number of carbon atoms of the alkyl group exceeds 10, the heat resistance of the obtained polyimide decreases in the use as a monomer of the polyimide. The number of carbon atoms of the alkyl group which can be selected as R⁴ is preferably 1 to 5 and more preferably 1 to 3, from the viewpoint that a higher heat resistance can be obtained when a polyimide is produced. In addition, the alkyl group which can be selected as R⁴ may be linear or branched.

R⁴ in the general formula (4) is more preferably a hydrogen atom or an alkyl group having 1 to 10 carbon atoms, from the viewpoint that a higher heat resistance can be obtained when a polyimide is produced. Especially, R⁴ in the general formula (4) is more preferably a hydrogen atom, a methyl group, an ethyl group, a n-propyl group, or an isopropyl group, and particularly preferably a hydrogen atom or a methyl group, from the viewpoints that the raw material is readily available and that the purification is easier.

The amount of the diene compound added is preferably 2 mole equivalents or more, and more preferably 2 to 10 mole equivalents to the Mannich base represented by the general formula (3). If the amount of the diene compound added is less than the lower limit, the yield of the bis(spiro norbornene) tends to be low. Meanwhile, if the amount of the diene compound added exceeds the upper limit, a by-product due to a side reaction tends to increase. Note that one of these diene compounds may be used alone, or two or more thereof may be used in combination.

In addition, in the second step, after the organic solvent, the base, and the diene compound are added to the reaction liquid, the Mannich base and the diene compound are reacted with each other by heating the obtained mixture liquid.

Any conditions can be employed for the heating, as long as the bis(spiro norbornene) represented by the general formula (5) can be produced by reacting the Mannich base and the diene compound with each other in the mixture liquid. A heating temperature for reacting the Mannich base and the diene compound with each other is preferably 30 to 180° C. (more preferably 50 to 140° C.). If the heating temperature is lower than the lower limit, the decomposition rate of the Mannich base tends to be low, so that the yield of the target product tends to be low. Meanwhile, if the heating temperature is above the upper limit, by-products such as vinyl ketone dimer and tetracyclododecene which is formed by Diels-Alder addition of another molecule of the diene to the target product tend to increase, so that the selectivity for the target product tends to be low.

In addition, a heating time for reacting the Mannich base and the diene compound with each other is preferably 0.01 to 10 hours, more preferably 0.01 to 7.0 hours, and further preferably 0.1 to 5.0 hours. If the heating time is less than the lower limit, the yield tends to be low. Meanwhile, if the heating time exceeds the upper limit, by-products tend to increase. Note that an atmosphere during the heating is preferably an inert gas atmosphere of nitrogen gas or the like from the viewpoints of coloring-prevention and safety.

In addition, as a method for the heating, it is possible to employ a method in which the mixture liquid of the Mannich base, the diene compound, the base, and the organic solvent is added dropwise to a reaction vessel preheated to the heating temperature. In addition, when the method in which the mixture liquid is added dropwise as described above is employed, a portion of the organic solvent may be placed in the reaction vessel in advance. This enables the reaction to be carried out more safely.

In addition, when an organic solvent having a boiling point lower than the heating temperature is used, a pressure container such as an autoclave may be employed. In this case, the heating may be started at normal pressure, or at a certain predetermined pressure. This allows various kinds of organic solvents to be used, and also enables reduction in thermal energy for solvent recycling.

Note that a pressure condition for the heating is not particularly limited, and is preferably 0.10 to 10 MPa, and more preferably 0.10 to 1.0 MPa. If the pressure condition is below the lower limit, the effect of reducing the thermal energy at the solvent recycling tends to be low. Meanwhile, if the pressure condition is above the upper limit, such a pressure condition tends to be difficult to achieve because of limitations of equipment.

By adding the organic solvent, the base, and the diene compound to the reaction liquid, and then heating the reaction liquid as described above, the 5-norbornene-2-spiro-α-cycloalkanone-α′-spiro-2″-5″-norbornene represented by the following general formula (5) can be obtained:

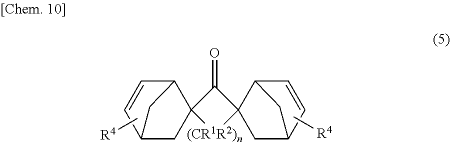

[R¹, R², and n in the formula (5) have the same meanings as those of R¹, R², and n in the formula (1), and R⁴s in the formula (5) have the same meaning as that of R⁴ in the formula (4)].

Note that R¹, R², and n in the general formula (5) have the same meanings as those of R¹, R², and n in the formula (1) (preferred ones thereof are also the same), and R⁴s in the general formula (5) have the same meaning as that of R⁴ in the formula (4) (preferred ones thereof are also the same). In addition, when multiple R¹s are present in the general formula (5) (when n is 2 or greater), the multiple R¹s may be the same or different, and are preferably the same from the viewpoints of ease of purification and the like. In addition, when multiple R²s are present in the general formula (5) (when n is 2 or greater), the multiple R²s may be the same or different, and are preferably the same from the viewpoints of ease of purification and the like. In addition, the multiple R⁴s in the general formula (5) may be the same or different and are preferably the same from the viewpoints of ease of purification and the like.

When the mixture liquid obtained by adding the organic solvent, the base, and the diene compound to the reaction liquid is heated to obtain the bis(spiro norbornene) in the present invention, an amine compound is first eliminated from the Mannich base represented by the general formula (3) under a neutral or basic condition, so that a compound having a bis(vinyl ketone) structure and being represented by the following general formula (6) is formed:

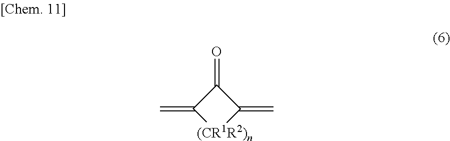

[R¹, R², and n in the formula (6) have the same meanings as those of R¹, R², and n in the formula (1)]. Subsequently, the compound having the bis(vinyl ketone) structure and the diene compound represented by the general formula (4) are reacted with each other by the so-called Diels-Alder reaction, so that the bis(spiro norbornene) represented by the general formula (5) is formed. In the present invention, the reaction is allowed to proceed under a neutral or basic condition as described above. Hence, the formation of by-products is suppressed at a higher level, and the bis(spiro norbornene) is produced more efficiently.

In addition, after the formation of the bis(spiro norbornene) by the reaction, the percentage of the compound having the bis(vinyl ketone) structure present in the mixture liquid after the reaction is preferably 2% by mole or less relative to the bis(spiro norbornene) (target product). If the percentage of the compound having the bis(vinyl ketone) structure present exceeds the upper limit, the target product tends to be colored, and the product tends to be viscous because of dimerization. Note that, from the viewpoint that the percentage of the compound having the bis(vinyl ketone) structure present is more surely made 2% by mole or less, it is preferable to set the content of the base to 2.0 to 5.0 mole equivalents to the acid contained in the reaction liquid, the heating temperature to 50 to 125° C., and the heating time to 0.5 to 10 hours in the second step.

In addition, after the formation of the bis(spiro norbornene) by the reaction, the percentage of the dimerization product (dimer), which is formed by dimerization of the compound having the bis(vinyl ketone) structure, present in the mixture liquid after the reaction is preferably 2% by mole or less relative to the bis(spiro norbornene) (target product). If the percentage of the dimer present exceeds the upper limit, the product tends to be viscous. Note that, from the viewpoint that the percentage of the dimer present is more surely made 2% by mole or less, it is preferable to set the content of the base to 2.0 to 5.0 mole equivalents to the acid contained in the reaction liquid, the heating temperature to 50 to 125° C., and the heating time to 0.5 to 10 hours in the second step. Note that the percentages of the compound having the bis(vinyl ketone) structure and the dimer which are present in the mixture liquid can be determined by the so-called HPLC analysis. As the apparatus used for the HPLC analysis and the like, a known apparatus and the like can be used as appropriate.

Note that, after the bis(spiro norbornene) represented by the general formula (5) is formed by the reaction, the bis(spiro norbornene) may be separated and taken out of the reaction liquid by performing any one or more of an extraction step, a purification step, and the like, as appropriate. Note that, after the formation of the bis(spiro norbornene) represented by the general formula (5) as described above, it is preferable to conduct a purification step from the viewpoint of obtaining the bis(spiro norbornene) with a higher purity and other viewpoints.

In addition, a method for separating and taking out the bis(spiro norbornene) from the mixture liquid after the reaction (an extraction step, a purification step, or the like), which is conducted after the formation of the bis(spiro norbornene) by the reaction, is not particularly limited, and a known method or the like can be employed as appropriate. For example, a crystallization method, a known extraction method as described in International Publication No. WO2011/099517, or the like may be employed, as appropriate.

As the method for separating and taking out the bis(spiro norbornene) from the mixture liquid after the reaction, it is more preferable to employ a crystallization method (to include a crystallization step) from the viewpoints that the purified bis(spiro norbornene) can be obtained more efficiently, and the ease and convenience of operation steps in a mass production scale are further improved. Specifically, as the step of purifying the bis(spiro norbornene), a crystallization method is preferably employed. A specific method for the crystallization is not particularly limited, and a known method can be employed as appropriate. For example, it is possible to employ, as appropriate, a crystallization method in which crystals are precipitated by cooling the mixture liquid after the reaction or the like. In addition, for the crystallization step, seed crystals may be used as appropriate.

A temperature condition and the like for the crystallization step vary depending on the kind of the target bis(spiro norbornene), and is not particularly limited. It is preferable to perform the crystallization by employing such conditions that the cooling is performed under a temperature condition of −25 to 25° C. (more preferably −20 to 0° C.) for 5 to 12 hours. If the temperature condition is above the upper limit, precipitation of crystals tends to be insufficient, and the yield tends to be low. Meanwhile, if the temperature condition is below the lower limit, the purity tends to be low because of precipitation of by-products.

Note that when the acidic solvent comprises a solvent (for example, methylcyclohexane or the like) in which the solubility of the bis(spiro norbornene) is high as the solvent, in addition to the formaldehyde derivative and the acid, it is preferable to perform a pretreatment step of removing the solvent in which the solubility of the bis(spiro norbornene) is high from the reaction liquid after the preparation of the bis(spiro norbornene) and then perform the crystallization of the bis(spiro norbornene), from the viewpoint of efficiently obtaining the bis(spiro norbornene) by the crystallization step. From such viewpoints, when the mixture liquid comprises a solvent in which the solubility of the bis(spiro norbornene) is high (for example, the first organic solvent or the like), it is preferable to include a pretreatment step of removing such a solvent. Note that the pretreatment step is not particularly limited, as long as the solvent (for example, the first organic solvent or the like) in which the solubility of the bis(spiro norbornene) is high can be removed by the method. A known method can be employed, as appropriate, for the pretreatment step. For example, a method in which the solvent is removed by azeotropic distillation with another component or the like may be employed, as appropriate.

In addition, in the pretreatment step, it is preferable to remove 60 to 100% by mass (more preferably 70 to 100% by mass) of the solvent (for example, the first organic solvent) in which the solubility of the bis(spiro norbornene) is high relative to the total amount of the solvent in which the solubility of the bis(spiro norbornene) is high contained in the reaction liquid (the mixture liquid after the reaction). If the removed amount (the removal ratio) is less than the lower limit, the amount of the product precipitated during the crystallization tends to decrease, so that the yield tends to be low. Note that the concentration of the solvent (for example, the first organic solvent) in which the solubility of the bis(spiro norbornene) is high in the reaction liquid (the mixture liquid after the reaction) from which the solvent (for example, the first organic solvent) in which the solubility of the bis(spiro norbornene) is high has been removed by the pretreatment step as described above is preferably 5% by mass or less (more preferably 3% by mass or less). If the concentration exceeds the upper limit, the amount of the product precipitated during the crystallization tends to decrease, so that the yield tends to be low. Note that, when the acidic solvent comprises the first organic solvent, it is more preferable to remove the first organic solvent at the above-described ratio (in the above-described removed amount) in the pretreatment step.

In addition, when the bis(spiro norbornene) is precipitated and separated after the reaction by crystallization from the mixture liquid in which the Mannich base and the diene compound have been reacted with each other, the solvent in the mixture liquid after the reaction is more preferably a solvent (poor solvent) in which the solubility of the bis(spiro norbornene) is low.

Note that the property of the solvent expressed by the phrase “in which the solubility of the bis(spiro norbornene) is low” herein is determined based on the solubility at 20° C. in the solvent mainly used for the reaction. In addition, as the solvent in which the solubility of the bis(spiro norbornene) low, a different solvent may be employed depending on the temperature condition for the crystallization. The solvent is not particularly limited, and may be one in which the solubility at 20° C. is lower than that in the solvent mainly used for the reaction. Especially, an organic solvent in which the solubility of the bis(spiro norbornene) greatly varies depending on the temperature is preferable, and an organic solvent in which the solubility of the bis(spiro norbornene) is 5% by weight or higher under a condition of 40 to 80° C., but is lower than 2% by weight under a condition of −20 to 0° C. is more preferable. The use of such a solvent enables the crystals to be precipitated more efficiently under a temperature condition of −25 to 25° C. (more preferably −20 to 0° C.). In addition, examples of the solvent in which the solubility of the bis(spiro norbornene) is low include methanol, ethanol, isopropanol, aqueous solutions thereof, and the like. Note that, when a solvent in which the solubility of the bis(spiro norbornene) is low is used as the organic solvent (second organic solvent) used in the second step, the solvent in the mixture liquid after the reaction may be replaced with the solvent in which the solubility of the bis(spiro norbornene) is low by removing the solvent (for example, the first organic solvent or the like) in which the solubility of the bis(spiro norbornene) is high in the pretreatment step, while leaving the organic solvent (second organic solvent) used in the second step.

According to the method for producing a 5-norbornene-2-spiro-α-cycloalkanone-α′-spiro-2″-5″-norbornene of the present invention, the bis(spiro norbornene) represented by the general formula (5) can be produced more efficiently in a more sufficient yield. Moreover, according to the method for producing a 5-norbornene-2-spiro-α-cycloalkanone-α′-spiro-2″-5″-norbornene of the present invention, the endo/exo ratio of the configuration of substituents in the bis(spiro norbornene) represented by the general formula (5) can be made 10/90 to 30/70 (more preferably 15/85 to 25/75). Note that, in the present invention, the bis(spiro norbornene) is produced in the second step by decomposing the Mannich base and simultaneously causing a Diels-Alder reaction. Here, when the heating temperature (reaction temperature) in the second step is set within the above-described preferred range (for example, 30 to 180° C.), the variable endo/exo ratio naturally falls in the above-described range. In addition, the bis(spiro norbornene) of the present invention has a ketone group, and the ketone group has priority in nomenclature. Hence, although the bis(spiro norbornene) is an endo adduct from the viewpoint of the reaction, the bis(spiro norbornene) obtained by the reaction is an exo isomer from the viewpoint of nomenclature.

In addition, the bis(spiro norbornene) represented by the general formula (5) obtained as described above can be preferably used as a raw material compound for producing an acid dianhydride monomer for polyimide production. Colorless transparent polyimides obtained by using the bis(spiro norbornene) as a starting raw material are particularly useful as materials for producing films for flexible printed wiring boards, heat resistant insulating tapes, enameled wires, protective coating agents for semiconductors, liquid crystal orientation films, transparent electro-conductive films for organic ELs, flexible substrate films, flexible transparent electro-conductive films, transparent electro-conductive films for organic thin-film solar cells, transparent electro-conductive films for dye-sensitized solar cells, flexible gas-barrier films, films for touch panels, interlayer insulating films, sensor substrates, transfer belts of printers, and the like. Furthermore, the bis(spiro norbornene) can be converted to a desired polymer or a cross-linked product by subjecting the bis(spiro norbornene) alone to a metathesis reaction, an addition polymerization, a radical polymerization, a cationic polymerization, an anionic polymerization, or the like. Moreover, if necessary, it is also possible to obtain a copolymer or a copolymerization cross-linked product by subjecting the bis(spiro norbornene) to a copolymerization reaction with any copolymerizable compound. In addition, an acid dianhydride obtained from the bis(spiro norbornene) is useful not only as a monomer for polyimides, but also as an epoxy-curing agent and a raw material for maleimides.

### EXAMPLES

Hereinafter, the present invention will be described more specifically based on Examples and Comparative Examples. However, the present invention is not limited to Examples below.

Note that, in the following description, the molecular structure of the compound obtained in each Example was identified by measuring IR and NMR spectra using IR measuring apparatuses (manufactured by JASCO Corporation under the trade names of FT/IR-460 and FT/IR-4100) and NMR measuring apparatuses (manufactured by VARIAN under the trade name of UNITY INOVA-600 and manufactured by JEOL Ltd. under the trade name of JNM-Lambda500).

### Example 1

### First Step

First, to a 1-L three-necked flask, 30.86 g (378.5 mmol) of dimethylamine hydrochloride was added. Next, to the three-necked flask, 12.3 g (385 mmol) of paraformaldehyde, 23.9 g (385 mmol) of ethylene glycol, and 12.95 g (154 mmol) of cyclopentanone were further added. Subsequently, 16.2 g (165 mmol) of methylcyclohexane was added to the three-necked flask, and then 0.4 g of 35% by mass hydrochloric acid (HCl: 3.85 mmol) was added to obtain a first mixture liquid. Note that the content of the acid (HCl) in the first mixture liquid was 0.025 mole equivalents to the ketone group of cyclopentanone (3.85 [the amount by mole of HCl]/154 [the amount by mole of cyclopentanone]=0.025).

Subsequently, the inside of the three-necked flask was purged with nitrogen, and the first mixture liquid was heated with stirring for 8 hours at normal pressure (0.1 MPa) with the temperature inside the three-necked flask being 85° C. Thus, a reaction liquid was obtained which contained a Mannich base which was a compound represented by the general formula (3), in which n was 2, R¹ and R² were all hydrogen atoms, and R³s were each a methyl group.

### Second Step

Next, the reaction liquid in the three-necked flask was cooled to 50° C., and then methanol (250 ml), 4.17 g of a 50% by mass aqueous dimethylamine solution (dimethylamine: 46.2 mmol), and 30.5 g (461.5 mmol) of cyclopentadiene were added to the reaction liquid in the three-necked flask to obtain a second mixture liquid. Subsequently, the inside of the three-necked flask was purged with nitrogen, and the second mixture liquid was stirred under heating at 65° C. for 5 hours at normal pressure (0.1 MPa) with the temperature inside the three-necked flask being 65° C. to form a compound. The thus obtained compound (5-norbornene-2-spiro-2′-cyclopentanone-5′-spiro-2″-5″-norbornene) in the reaction liquid was quantified by high performance liquid chromatography to find that the reaction yield was 76%.

Subsequently, the second mixture liquid in the three-necked flask was concentrated by azeotropic distillation of methylcyclohexane and methanol to remove 100 mL of liquid from the second mixture liquid. Note that by removing the 100-mL portion of the liquid, most methylcyclohexane (75% by mass of the total amount of methylcyclohexane in the second mixture liquid before the concentration) was removed from the second mixture liquid. Next, the second mixture liquid from which methylcyclohexane had been removed was cooled under a temperature condition of −20° C. for 12 hours to precipitate crystals, and then the crystals were obtained by vacuum filtration. The thus obtained crystals were subjected to a washing step with 20 mL of methanol at −20° C. three times, and then methanol was removed by vaparization. Thus, 17.4 g of the compound (5-norbornene-2-spiro-2′-cyclopentanone-5′-spiro-2″-5″-norbornene) was obtained (final yield: 47%).

To confirm the structure of thus obtained compound, IR and NMR (¹H-NMR and ¹³C-NMR) measurements were conducted. As a result, the compound was confirmed to be 5-norbornene-2-spiro-2′-cyclopentanone-5′-spiro-2″-5″-norbornene represented by the following general formula (7):

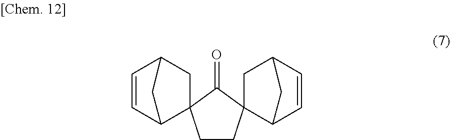

Note that the ratio (endo/exo) between the endo isomer and the exo isomer was found to be 10/90.

### Example 2

5-Norbornene-2-spiro-2′-cyclopentanone-5′-spiro-2″-5″-norbornene was obtained in the same manner as in Example 1, except that the amount of 35% by mass hydrochloric acid used was changed from 0.4 g (HCl: 3.85 mmol) to 0.2 g (HCl: 1.93 mmol). Note that the content of the acid (HCl) in the first mixture liquid was 0.0125 mole equivalents to the ketone group of cyclopentanone. In addition, the structure of the compound was analyzed in the same manner as in Example 1. As a result, the obtained compound was confirmed to be 5-norbornene-2-spiro-2′-cyclopentanone-5′-spiro-2″-5″-norbornene, and the reaction yield was 70%.

### Example 3

5-Norbornene-2-spiro-2′-cyclopentanone-5′-spiro-2″-5″-norbornene was obtained in the same manner as in Example 1, except that the amount of 35% by mass hydrochloric acid used was changed from 0.4 g (HCl: 3.85 mmol) to 1.1 g (HCl: 10.8 mmol). Note that the content of the acid (HCl) in the first mixture liquid was 0.070 mole equivalents to the ketone group of cyclopentanone. In addition, the structure of the compound was analyzed in the same manner as in Example 1. As a result, the obtained compound was confirmed to be 5-norbornene-2-spiro-2′-cyclopentanone-5′-spiro-2″-5″-norbornene, and the reaction yield was 71%.

### Example 4

5-Norbornene-2-spiro-2′-cyclopentanone-5′-spiro-2″-5″-norbornene was obtained in the same manner as in Example 1, except that the amount of 35% by mass hydrochloric acid used was changed from 0.4 g (HCl: 3.85 mmol) to 0.8 g (HCl: 7.7 mmol). Note that the content of the acid (HCl) in the first mixture liquid was 0.050 mole equivalents to the ketone group of cyclopentanone. In addition, the structure of the compound was analyzed in the same manner as in Example 1. As a result, the obtained compound was confirmed to be 5-norbornene-2-spiro-2′-cyclopentanone-5′-spiro-2″-5″-norbornene, and the reaction yield was 70%.

### Comparative Example 1

5-Norbornene-2-spiro-2′-cyclopentanone-5′-spiro-2″-5″-norbornene was obtained in the same manner as in Example 1, except that the amount of 35% by mass hydrochloric acid used was changed from 0.4 g (HCl: 3.85 mmol) to 3.2 g (HCl: 30.8 mmol). Note that the content of the acid (HCl) in the first mixture liquid was 0.20 mole equivalents to the ketone group of cyclopentanone. In addition, the structure of the compound was analyzed in the same manner as in Example 1. As a result, the obtained compound was confirmed to be 5-norbornene-2-spiro-2′-cyclopentanone-5′-spiro-2″-5″-norbornene, and the reaction yield was 27%.

### Comparative Example 2

5-Norbornene-2-spiro-2′-cyclopentanone-5′-spiro-2″-5″-norbornene was obtained in the same manner as in Example 1, except that the amount of 35% by mass hydrochloric acid used was changed from 0.4 g (HCl: 3.85 mmol) to 1.28 g (HCl: 12.3 mmol). Note that the content of the acid (HCl) in the first mixture liquid was 0.080 mole equivalents to the ketone group of cyclopentanone. In addition, the structure of the compound was analyzed in the same manner as in Example 1. As a result, the obtained compound was confirmed to be 5-norbornene-2-spiro-2′-cyclopentanone-5′-spiro-2″-5″-norbornene, and the reaction yield was 60%.

### Comparative Example 3

5-Norbornene-2-spiro-2′-cyclopentanone-5′-spiro-2″-5″-norbornene was obtained in the same manner as in Example 1, except that the amount of 35% by mass hydrochloric acid used was changed from 0.4 g (HCl: 3.85 mmol) to 1.6 g (HCl: 15.4 mmol). Note that the content of the acid (HCl) in the first mixture liquid was 0.10 mole equivalents to the ketone group of cyclopentanone. In addition, the structure of the compound was analyzed in the same manner as in Example 1. As a result, the obtained compound was confirmed to be 5-norbornene-2-spiro-2′-cyclopentanone-5′-spiro-2″-5″-norbornene, and the reaction yield was 61%.

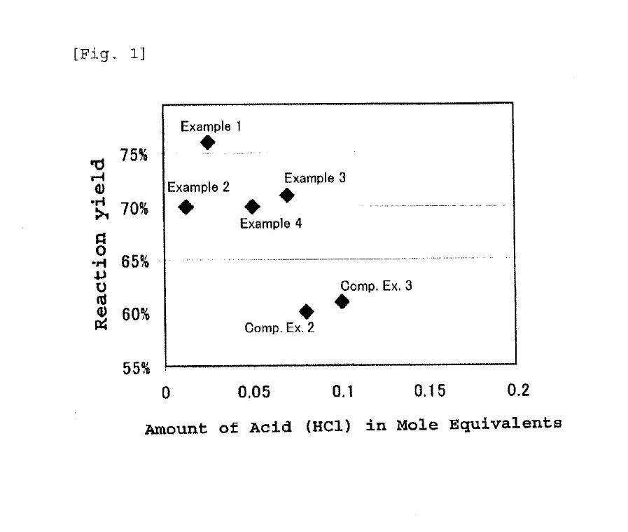

Table 1 shows the result of the reaction yield of the product obtained in each of Examples 1 to 4 and Comparative Examples 1 to 3 and the content (mole equivalents) of the acid (HCl) in the first mixture liquid. FIG. 1 shows a graph showing the relationship between the reaction yield of the product obtained in each of Examples 1 to 4 and Comparative Example 2 and 3 and the content (mole equivalents) of the acid (HCl) in the first mixture liquid used for producing the product.

TABLE 1

| | Amount of acid (HCl) in | |
|---|---|---|
| | mole equivalents | Reaction yield |
| Example 1 | 0.025 | 76% |
| Example 2 | 0.0125 | 70% |
| Example 3 | 0.070 | 71% |
| Example 4 | 0.050 | 70% |
| Comp. Ex. 1 | 0.20 | 27% |
| Comp. Ex. 2 | 0.080 | 60% |
| Comp. Ex. 3 | 0.10 | 61% |

As is apparent from the results shown in Table 1 and FIG. 1, it was found that 5-norbornene-2-spiro-2′-cyclopentanone-5′-spiro-2″-5″-norbornene was obtained in an extremely high reaction yield of 70% or higher in each of the cases (Examples 1 to 4), where the content of the acid (HCl) was adjusted to the range from 0.010 to 0.075 mole equivalents to the ketone group of the carbonyl compound.

### INDUSTRIAL APPLICABILITY

As described above, according to the present invention, it is possible to provide a method for producing a 5-norbornene-2-spiro-α-cycloalkanone-α′-spiro-2″-5″-norbornene, the method making it possible to more efficiently produce the 5-norbornene-2-spiro-α-cycloalkanone-α′-spiro-2″-5″-norbornene in a higher yield.

Accordingly, the method for producing a 5-norbornene-2-spiro-α-cycloalkanone-α′-spiro-2″-5″-norbornene of the present invention is especially useful as, for example, a method for producing a raw material compound (raw material monomer) for producing polyimides for flexible printed wiring boards, polyimides for heat resistant insulating tapes, polyimides for enameled wires, polyimides for protective coatings of semiconductors, polyimides for liquid crystal orientation films, polyimides for transparent electrode substrates of organic ELs, polyimides for transparent electrode substrates of solar cells, polyimides for transparent electrode substrates of electronic papers, materials for various gas barrier film substrates, polyimides for interlayer insulating films, polyimides for sensor substrates, polyimides for printer transfer belts, where heat resistance is required, and the like.

1. A method for producing a 5-norbornene-2-spiro-α-cycloalkanone-α′-spiro-2″-5″norbornene, comprising:
a first step of forming a Mannich base by reacting a carbonyl compound and an amine compound with each other in an acidic solvent, to thereby obtain a reaction liquid comprising the Mannich base in the acidic solvent,
the acidic solvent comprising a formaldehyde derivative and an acid represented by a formula: HX (in the formula, X represents one selected from the group consisting of F, Cl, Br, I, CH₃COO, CF₃COO, CH₃SO₃, CF₃SO₃, C₆H₅SO₃, CH₃C₆H₄SO₃, HOSO₃, and H₂PO₄),
the carbonyl compound being represented by the following general formula (1): 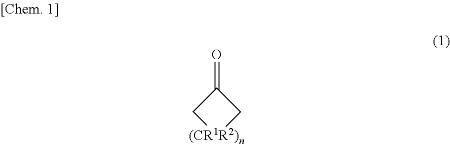
[in the formula (1), R¹ and R² each independently represent one selected from the group consisting of a hydrogen atom, alkyl groups having 1 to 10 carbon atoms, and a fluorine atom, and n represents an integer of 0 to 12],
the amine compound being represented by the following general formula (2): 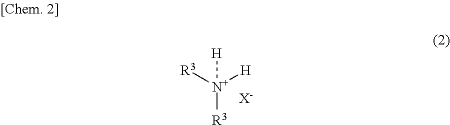
[in the formula (2), R³s each independently represent one selected from the group consisting of linear chain saturated hydrocarbon groups having 1 to 20 carbon atoms, branched chain saturated hydrocarbon groups having 3 to 20 carbon atoms, saturated cyclic hydrocarbon groups having 3 to 20 carbon atoms, and saturated hydrocarbon groups having a hydroxyl group and 1 to 10 carbon atoms, the two R³s may be bonded to each other to form a ring selected from the group consisting of a pyrrolidine ring, a piperidine ring, a piperazine ring, and a morpholine ring, and X⁻ represents one selected from the group consisting of F⁻, Cl⁻, Br⁻, I⁻, CH₃COO⁻, CF₃COO⁻, CH₃SO₃⁻, CF₃SO₃⁻, C₆H₅SO₃⁻, CH₃C₆H₄SO₃⁻, HOSO₃⁻, and H₂PO₄⁻],
the Mannich base being represented by the following general formula (3): 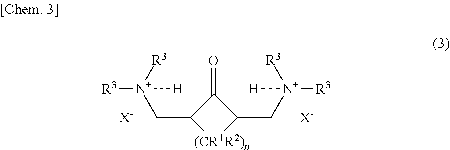
[R¹, R², and n in the formula (3) have the same meanings as those of R¹, R², and n in the formula (1), and R³s and X⁻s in the formula (3) have the same meanings as those of R³s and X⁻ in the formula (2)]; and
a second step of reacting the Mannich base and a diene compound with each other by adding an organic solvent, a base in an amount of 1.0 to 20.0 mole equivalents to the acid, and the diene compound to the reaction liquid, and then heating the reaction liquid, to thereby form a 5-norbornene-2-spiro-α-cycloalkanone-α′-spiro-2″-5″-norbornene,
the diene compound being represented by the following general formula (4): 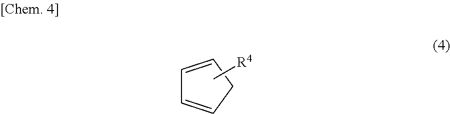
[in the formula (4), R⁴ represents one selected from the group consisting of a hydrogen atom, alkyl groups having 1 to 10 carbon atoms, and a fluorine atom],
the 5-norbornene-2-spiro-α-cycloalkanone-α′-spiro-2″-5″-norbornene being represented by the following general formula (5): 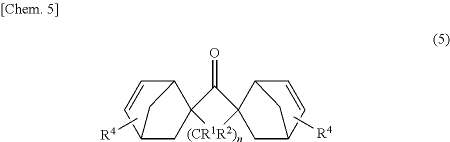
[R¹, R², and n in the formula (5) have the same meanings as those of R¹, R², and n in the formula (1), and R⁴s in the formula (5) have the same meaning as that of R⁴ in the formula (4)], wherein
a content of the acid in the acidic solvent used in the first step is 0.01 to 0.075 mole equivalents to the ketone group of the carbonyl compound.

2. The method for producing a 5-norbornene-2-spiro-α-cycloalkanone-α′-spiro-2″-5″-norbornene according to claim 1, wherein
the content of the acid in the acidic solvent used in the first step is 0.01 to 0.070 mole equivalents to the ketone group of the carbonyl compound.
